(19) United States
(12) Patent Application Publication
(10) Pub. No.: US 2026/0034359 A1
(43) Pub. Date: Feb. 5, 2026

(54) SYSTEMS AND METHODS FOR THE PREVENTION AND TREATMENT OF SUDDEN DEATH SYNDROMES

(71) Applicant: Daniel Rubens, Seattle, WA (US)

(72) Inventor: Daniel Rubens, Seattle, WA (US)

(21) Appl. No.: 19/247,636

(22) Filed: Jun. 24, 2025

Related U.S. Application Data
(60) Provisional application No. 63/677,790, filed on Jul. 31, 2024.

(51) Int. Cl. A61N 1/36; A61M 21/00; A61N 1/04

(52) U.S. Cl. CPC A61N 1/36036; A61M 21/00; A61N 1/0408; A61N 1/0484; A61N 1/36031; A61N 1/36034; A61M 2021/0027

(57) ABSTRACT

A system for the prevention of a sudden death syndrome in a user includes a galvanic vestibular stimulator and an audio vestibular stimulator. The system is used in a method for preventing permanent breathing arrest in a user susceptible to a sudden death syndrome by stimulating a vestibular system of the user via at least one of electrical galvanic vestibular stimulation and sound-induced vestibular stimulation using bone conduction.

### CROSS-REFERENCE TO RELATED APPLICATION

This application claims benefit to U.S. Provisional Application No. 63/677,790, filed Jul. 31, 2024, which is hereby incorporated by reference herein in its entirety.

### BACKGROUND OF THE INVENTION

### 1. Field of the Invention

The present disclosure relates to systems for the prevention and treatment of medical conditions. More particularly, the disclosure relates to systems for the prevention and treatment of respiratory arrest in individuals prone to sudden death syndromes.

### 2. State of the Art

Three fatal human syndromes bear important similarities: Sudden Infant Death Syndrome (SIDS), Sudden Unexplained Death in Children (SUDC) and Sudden Unexplained Death in Epilepsy (SUDEP). All three of these sudden death syndromes pertain to individuals who succumb most commonly during a sleep period and are frequently found in the prone position. Apnea and respiratory arrest are believed to be common pathophysiological elements in these syndromes.

Various systems have been developed to monitor individuals prone to sudden death syndromes, e.g., SUDEP, and to attempt to provide alerts to the subject individuals or to caregivers, e.g., that a seizure was detected. Systems have also been provided to stimulate a sleeping individual in an attempt to interrupt various dangerous physiological conditions, e.g., by waking the individual, and restore a stable condition. However, it is theorized by the applicant that the correct causes of the various sudden death syndromes have not previously been comprehensively identified, and/or the proper stimuli have not been applied to break the conditions that lead to potentially lethal results. Further, none of the prior proposed stimulation systems permit an individual to continue sleeping while an effective prophylactic stimulation is applied to the individual to prevent or reduce the risk of death.

The diving reflex is a powerful primordial response the origin of which is to protect all mammals including humans from drowning. The diving reflex response includes apnea, bradycardia and increased peripheral vascular resistance. Apnea prevents inhalation of water while submerged. Bradycardia is a slowed heart rate, generally under 60 beats per minute. An increased vascular resistance reduces blood circulation to the extremities and maximizes the availability of oxygenated blood for the heart and brain. Normally, the diving reflex requires submersion of the face or head in order to be activated.

Once the diving reflex is activated in a submerged individual, the individual can remain apneic while underwater for an extended period. The diving reflex is especially prevalent in early human childhood, occurring in 100% of infants up to 6 months of age and 90% of infants up to one year. The diving reflex also frequently occurs in humans at any age in response to profound hypoxia from any cause. As one example, seizure activity in adult animals has been demonstrated to evoke diving reflex apnea.

Splashing cold water onto the face can initiate the diving reflex, in expectation of submersion. There is however no clear description in the relevant literature as to the mechanism underlying how diving reflex apnea persists for an extended time when an infant (or other mammal) remains submerged for a prolonged period. The persistence of induced apnea during submersion is an extremely powerful response if one considers that it counters the regulated breathing efforts that occur automatically and continuously throughout life when one is breathing air.

### SUMMARY OF THE INVENTION

It is proposed by applicant that a key factor triggering the maintenance of apnea/vocal cord closure in the submerged individual is the sustained increase in barometric pressure associated with submersion compared to the ‘atmospheric-air’ state. Supportive evidence emanates from the finding that diving reflex bradycardia is increasingly more intense at deeper depths of submersion.

Many individuals experience hypoxic events during sleep and the vast majority survive. A great number of adults have both undiagnosed and diagnosed sleep apnea that is not properly treated without succumbing to death each sleep period. Nevertheless, the sleep state is an important risk factor in individuals susceptible to sudden death and shares important corollaries with being submerged. During sleep, there is suppression of sensory inputs comparable to that which occurs with submersion underwater. It is commonly dark, sounds are suppressed, and there is decreased or absent activation of the vestibular system to maintain balance.

For fatality to occur, the applicant proposes two distinct prerequisites. First, a precipitating event during sleep such as a seizure or apneic episode that results in significant hypoxia. Second, this hypoxia is followed by a sustained diving reflex response. This occurs in order to protect the individual from drowning due to the misperception by the brain that the individual is continually submerged. This can occur during sleep when the pressure around the brain or inner and middle ear reaches a threshold similar to that which occurs when an individual's head is submerged underwater. A prolonged diving response is no longer protective and unremitting respiratory arrest can lead to fatality.

The vestibular system is a proposed pathway whereby barometric pressure influences brain activity. Vestibulo-cochlear hair cells (VCHC's) are unique in that their activity is responsive to changes in barometric and intracranial pressure (ICP). Importantly, vestibular hair cell activation has been identified to trigger respiration and hypoxic movement arousal. Seizures surrounding sudden death syndromes have been identified to elicit substantial increases in intra-cranial pressure. Consequently, sudden death syndromes might eventuate following a prolonged seizure or other cause of apnea, where there is concurrent sustained increase in intracranial pressure that matches the pressure of submersion. In such case, the diving reflex could be unremitting and apnea would persist whether or not terminal gasp attempts are initiated. The prone sleeping position increases abdominal and intra-thoracic pressure with a consequent increase in intracranial pressure. This is particularly significant in infants and children that have compressible cartilaginous rib cages.

Children and infants in particular, are prone to fluid accumulation and infection in the middle ear. Inflammation and positive viral antigens have been identified in the inner and middle ears of SIDS cases. Middle ear fullness with increased pressure and further suppression of sound can increase a misperception by the brain during sleep that the individual is continually submerged. Consequently, this may be an important risk factor for persistent respiratory arrest in infants that are innately driven to powerfully activate a diving response to hypoxia.

Apnea during infancy and early childhood is common especially in the presence of an upper respiratory tract infection. Notably, it has been reported that more than 60% of SIDS cases are known to have experienced a mild to moderate upper respiratory infection in the days prior to death. The significance of this finding may relate to a higher proclivity for hypoxic events.

In addition, neuro-histopathology findings from a series of SIDS and SUDC cases identified hippocampal dentate gyrus abnormalities in 70% of victims aged 3 weeks to 16 years, suggesting seizure activity as an important commonality across all sudden death syndrome cases. Such lesions are classically associated with temporal lobe epilepsy which is notable as being highly represented in SUDEP. The majority of SUDEP cases occur immediately following a seizure. A recent study identified that many children succumbing to SUDC experienced a seizure in the lead up to fatality.

Overt motor signs of a seizure are rarely, if ever, seen in infancy, but seizure activity may still be present. This would explain why overt motor seizure activity has not been reported during witnessed SIDS events. Literature further reports an example of a near-miss SUDEP case without convulsive movements, but with seizure-associated obstructive apnea, in an adult. See Lacuey N. et al., Ictal laryngospasm monitored by video-EEG and polygraphy: a potential SUDEP mechanism. Epileptic Disord. 2018 Apr. 1;20 (2): 146-150.

Rodent models are considered relevant for the study of sudden death syndromes in humans. Rat models of SUDEP have been used to demonstrate that seizure activity drives massive increases in recurrent laryngeal nerve activity with a resultant obstructive apnea due to laryngospasm. Further, in such rat models, electromyograph evidence of inspiratory effort and electrocardiogram evidence of brady-arrhythmia support the conclusion that obstructive apnea is present in SUDEP cases. These findings lead to an overall sequence of events from seizure commencement through obstructive apnea, respiratory arrest, cardiac failure and death. In the SUDEP context, breath attempts continue from the start of obstructive apnea to a point designated “respiratory arrest” or “terminal apnea.”

While it has been argued that the cessation of breaths or breath attempts in SUDEP may be due to brainstem shutdown or failure, applicant relies upon a model of events in sudden death syndromes in which brainstem shutdown or failure is not the direct cause.

Respiratory arrest (also known as breathing arrest), or the onset of terminal apnea, is considered to be the onset of the diving response and prolongs the apneic period well beyond the duration of the seizure. It is recognized that persistent apnea in the presence of severe peri-ictal and post-ictal hypoxia can ultimately be fatal.

Therefore, a method and system to prevent or interrupt persistent apnea is provided. The system is adapted to be easily applied and removed. The system uses non-implantable sensors and stimulators.

The method may be optionally implemented in conjunction with appropriate sensors and detectors that detect respiratory arrest or seizure activity. In an embodiment, the method and system may detect persistent respiratory arrest using various detectors and sensors including, but not limited to, respiratory effort biomarkers and movement sensors. Respiratory effect biomarkers include, but are not limited to, EMG, seizure activity biomarkers such as EEG sensors, oxygen status sensors such as pulse oximeters or near infrared spectroscopy sensors, otoacoustic emission sensors as an indication of intracranial pressure, transcutaneous carbon dioxide sensors, temperature sensors, electrodermal activity sensors, and vestibular evoked myogenic potential (VEMP) measurement used to assess the function of the otolithic organs of the inner ear. Movement sensors include, but are not limited to, gyroscopes and accelerometers, breathing sensors, and chest wall and/or abdominal expansion/contraction detectors.

The method and system prevent or interrupt persistent apnea using stimuli that can be applied to a user, worn by the user, and operated to have an effect on the user, while the user sleeps. The stimuli are adapted to stimulate the vestibular system in a manner that replicates vestibular system activity in an awake individual.

The method and system direct a stimulator worn by the user to prevent or treat persistent respiratory arrest including reversing or preventing vocal cord closure.

The method and system are directed to stimulate the vestibular system of the user to prevent initiating the diving response or reverse the effects thereof. The effect of such a reversal is restoration of breathing effort and, provided the airway is clear, effective restoration of respiration.

The method and system include one or more options to activate the vestibular system, including galvanic vestibular stimulation and bone conduction of low frequency sound which stimulates the vestibular system. Such methods provide effective activation of the vestibular system to ideally prevent respiratory arrest as well as, if necessary, restart breathing after the point of respiratory arrest. The system may include an oxygen delivery system, and the method may include delivery of oxygen to one or both of prevent breathing arrest or restart arrested breathing.

In embodiments, the stimulators operate non-invasively to apply stimulation via bone conduction to induce vestibular stimulation. In embodiments, the stimulators are adapted to be temporarily seated adjacent the mastoid process or another skull bone near or forming the external auditory meatus. In embodiments, the stimulators may be incorporated into gel or adhesive pads positioned on or adjacent the mastoid process or gel or adhesive pads or ear plugs positioned on or adjacent a bone inside the external auditory meatus. The gel or adhesive pads or ear plugs can be coupled to or incorporated into a headband or wearable head cap. Such headband or cap may also include any optional sensors for directly or indirectly detecting respiratory arrest.

The system and use thereof is adapted to prevent or reverse temporary or terminal apnea events including respiratory arrest. In embodiments, the method and use of the system is implemented without reliance upon sensors that detect respiratory arrest, and a level of vestibular stimulation is provided continuously or at regular intervals to prevent or overcome respiratory arrest. The level of stimulation is preferably set at a level that permits the user to sleep while receiving the stimulation while obtaining the preventative or treatment benefits.

Then, if breathing arrest is detected, notwithstanding any prophylactic treatment already being provided, suitable vestibular stimulation is given to the user to restart breathing. If the user is already receiving a baseline of prophylactic treatment, the secondary treatment stimulation on breathing arrest detection is higher than the baseline stimulation.

The described rescue method consequently provides information to the brain to combat the two prerequisites that lead to sudden death. This is achieved by precipitating powerful additional input to respiratory drive comparable to that which occurs with vestibular activity in the awake state. This is also similar to the awake state being protective of apnea events in those that suffer from sleep apnea.

### BRIEF DESCRIPTION OF THE DRAWINGS

FIG. 1 is a schematic diagram of an embodiment of a system for detection and stimulation for the prevention of sudden death syndromes.

FIG. 2 is a schematic diagram of an embodiment of a system for stimulation for the prevention of sudden death syndromes.

FIG. 3 is an illustration of a wearable system according to FIG. 1 or 2, as worn around the head of a user.

FIG. 4 is a chart of control and experimental data of one rat experiment using sound vestibular stimulation.

FIG. 5 is a logarithmic plot of chart of the effect of sound stimulus frequency on breathing rate.

FIG. 6 is a chart of experimental data of one rat experiment using galvanic vestibular stimulation.

### DETAILED DESCRIPTION OF THE PREFERRED EMBODIMENTS

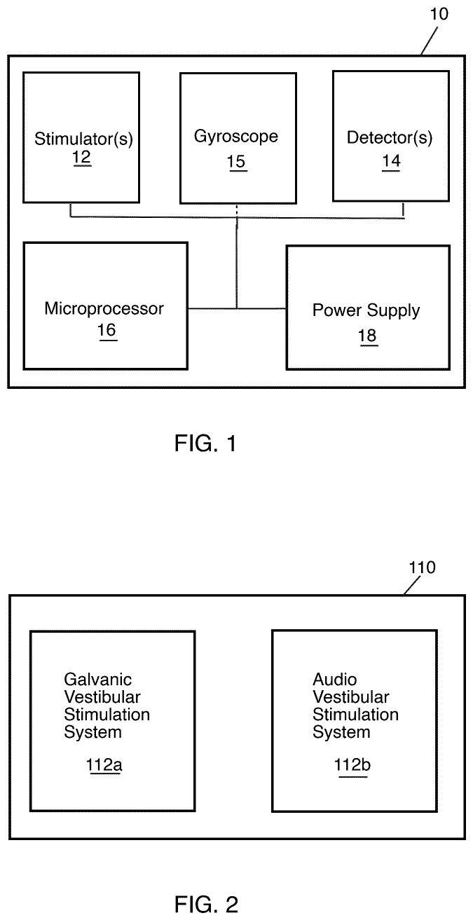

Turning now to FIG. 1, a system 10 for detection and stimulation to detect and prevent sudden death syndromes is shown. In an embodiment, the system 10 includes one or more stimulators 12, optionally one or more detectors 14, a microprocessor system 16 for processing input at the detectors 14 and controlling the stimulators 12, and a power supply 18 to power the stimulators, detectors, and microprocessor systems. The system 10 may be contained in a small package. The power supply 18 may be a DC battery or an AC source.

The one or more stimulators 12 are adapted to stimulate the vestibular system. In an embodiment, a stimulator 12 includes a current source coupled with a pulse/ramp/wave generator to create an alternating or sinusoidal stimulus train applied through a pair of galvanic dermal electrode pads operating as an anode and cathode. The current source can preferably be adjusted to modify one or more of the amplitude, frequency, and waveform of the current signal. Each pad includes a conductive gel or adhesive sized and adapted to be positioned externally on the skin, preferably over the mastoid process. The location permits a galvanic signal to be transmitted by conductive dermal electrodes to the vestibular system. No implantation of the stimulators is required.

In an embodiment, the stimulator 12 may include a sound wave generator, at least one sound/vibration transducer, and preferably a pair of sound transducers, which together are adapted to generate and transmit audio signals to a user of the stimulator. The transducers may include gel or adhesive pads adapted to be positioned externally on the skin over the mastoid process or as ear pads or plugs adapted to be inserted fully or partially into the ear canal to seat adjacent the bone component inside the external auditory meatus such that the sound waves can be transferred via bone conduction to the vestibular system of the user. The sound wave generator is preferably adapted to generate audio signals below 250 Hz, optionally down to 50 Hz, and may include infrasonic frequencies. The sound wave generator can also generate frequencies above 250 Hz up to 20,000 Hz, preferably at a dB intensity that will not awaken the individual.

In another embodiment, the system includes both a galvanic stimulation system and an audio signal generator such that both stimuli can be provided in combination.

The detectors 14 are adapted to detect indications of breathing arrest. The detectors may include, by way of example only and not by way of limitation, biomarkers and movement detectors.

The biomarker detectors can include, but are not limited to, electromyography (EMG) sensors to detect signals of motor nerves, seizure activity biomarkers such as electroencephalogram (EEG) sensors to sense brain activity, oxygen status sensors such as pulse oximeters or near infrared spectroscopy sensors to detect blood oxygen levels, otoacoustic emission sensors to indicate intracranial pressure, transcutaneous carbon dioxide sensors, temperature sensors, and vestibular evoked myogenic potential (VEMP) sensors to assess the function of the otolithic organs of the inner ear and vestibular activity.

By way of example, thoracic EMG signals can be detected at by sensors 14 from the head-worn system 10, or from other sensors at or displaced from the thorax and which correspond to the increased respiratory effort resulting from when the airway is occluded. See, e.g., U.S. Pat. No. 11,844,605, entitled “System, Method and Biomarkers for Airway Obstruction” to Stewart et al., and which is incorporated by reference herein in its entirety. See, also, Stewart et al., “Obstructive Apnea Due To Laryngospasm Links Ictal To Postictal Events In SUDEP Cases And Offers Practical Biomarkers For Review Of Past Cases And Prevention Of New Ones”, Epilepsia, 58 (6): e87-e90, 2017.

By way of another example, the sensing of VEMPs can be used to trigger a stimulation response, as discussed below. In addition, VEMPs can be used to setup and personalize the system for a specific user, without requiring the user to experience an apneic episode. To that end, an individual's response to a stimulation from the system can be measured by detecting VEMPs. The detection of VEMPs can confirm both that the system is functioning for the user and that the sensitivity of the system and any stimulation signal is appropriate. Adjustment can then be made, if appropriate.

The movement sensors can include, by way of example only, gyroscopes 15 or accelerometers, breathing sensors, and chest wall and/or abdominal expansion/contraction detectors. In an embodiment, a gyroscope 15 can be used to detect changes in an individual wearer's head position and, in response, activation of stimulation from the stimulators 12 in a feedback loop. The gyroscope 15 provides movement, balance, and vestibular input into the system. More specifically, the gyroscope can identify when the system wearer's head is in a prone position. Many individuals, particularly children, suffering a sudden death event under a relevant syndrome are positioned in a prone position when found. When the wear is identified as being in the prone position, such feedback can be used to activate one or more of the stimuli described herein to cause the individual to move out of the prone position or to interrupt current physiological conditions and thereby prevent the sudden death syndrome from occurring. Further, the lack of movement is also correlated to an apnea. Therefore, the detection of movement, or lack thereof, by way of the gyroscope or other body-worn sensors, can be used as an indirect measure of breathing cessation, and optionally provide the wearer feedback stimuli to breathe. Alternatively, the position of the wearer's head, as determined from the gyroscope or other body sensors, can be recorded and provide information to a physician or pathologist to assist in identifying circumstances leading to a fatality or other medical issue.

The data from the sensors 14, 15 may be fed to the microprocessor 16, which operates to make decision and, as appropriate, trigger the stimulators 12 at an appropriate level of activation upon sensed data indicating respiratory arrest. The sensors 14, 15 and the stimulators 12 may utilize the same electrodes, such that only a single set of electrodes is required, which operates in either a sensing or stimulating state depending on the present requirements of the system. The indicating event can be based on one or more various markers or levels dropping below or exceeding predetermined thresholds stored in memory associated with the microprocessor. Alternatively, the indicating event can be based on intelligent analysis by the microprocessor using an algorithm or algorithms that are trained from one or more training data sets. The detectors are used and operational in an embodiment of the system that actuates the stimulators to stimulate the vestibular system when the predetermined threshold data or machine training intelligence indicates that respiratory arrest has occurred or is about to occur. Further, the system can be used such that the stimulators 12 regularly provide a first level of prophylactic background stimulation that is intended to stimulate the vestibular system to prevent breathing arrest from occurring. However, should breathing arrest occur as detected by the detectors 14, then the stimulation from the stimulators 12 can be elevated to a second level intended to further stimulate the user's vestibular system and arouse the user's from an arrested breathing state. In another manner of operation, the stimulus can be applied by the stimulators 12 only if the biomarker, movement, or other associated signal is identified for a specific period of time, for example, occurring for greater than 5 seconds.

Turning to FIG. 2, it is recognized that for various individuals it may be appropriate to operationally use the system 110 in an embodiment without the detectors and potentially without the costs associated with complex data processing. Such a system 110 would necessarily include only the preferred galvanic stimulator 112a and/or audio stimulator 112b, along with any power supply (not shown). The microprocessor control 16 (FIG. 1) would then be optional as well.

In the embodiment of 110, the respective stimulators 112a, 112b would be set to provide consistent or regular stimulation to the wearing user while the user is in a sleep state in order to prevent respiratory arrest, or if it does occur to provide sufficient and regular stimulation to arouse the user from respiratory arrest before a terminal event. In such a use case, the system can be adapted to stimulate the user constantly or at regular intervals. By way of example, regular intervals may be timed at every 0.5 seconds, 1 second, 30 seconds, or 1 minute, or any other suitable interval, such that were respiratory arrest to occur between stimulation, the user could be aroused prior to a terminal respiration arrest event.

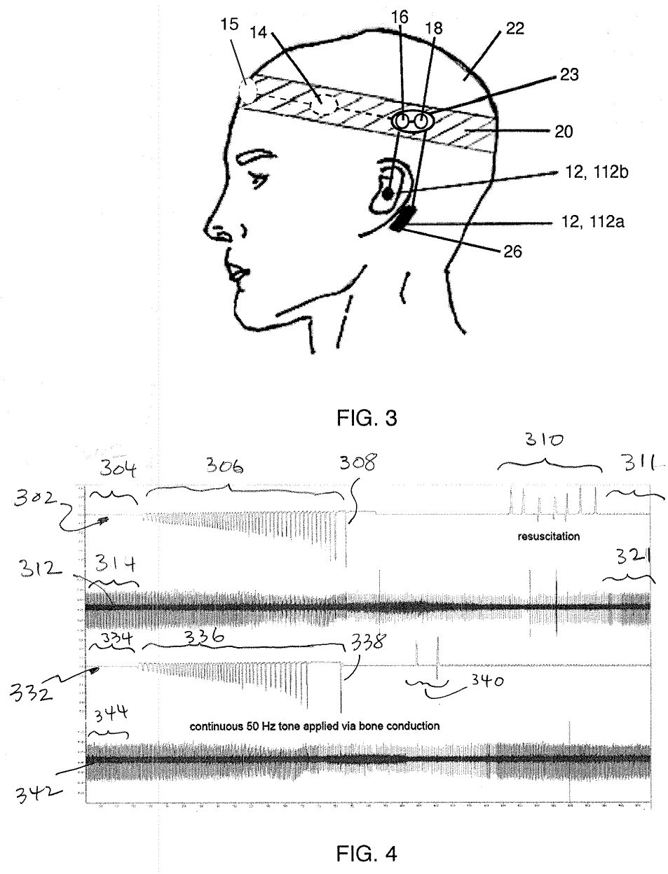

Referring now to FIG. 3, the systems 10, 110 may be implemented in a headband 20 wearable on a head 22 of a user. The headband 20 may be elastic or secured on the head with hook and loop fasteners or other size adjustable closures. The headband preferably includes one or more pockets 23 adapted to retain all the appropriate components. The stimulators 12 are arranged relative to the headband 20, and the headband is adapted to be worn in a manner that presents the electrodes and/or transducers for the stimulators 12 at or near the ears or against the bones that will conduct the stimulus to the vestibular system of the user. Pad components 26 of the stimulators 12 may be attached to the skin of the user with an adhesive. The stimulators may be further or alternatively retained against the user's skin by the elasticity of the headband applying compression against the pad. The pads may be retained in the form of an earplug fully or partially seated within the ear canal. The pads coupled to the galvanic stimulator are preferably adapted to be seated during use adjacent the mastoid process. The pads coupled to the audio stimulator are preferably adapted to be seated during use partially in the ear inside the external auditory meatus. However, the same pads may be utilized for applying both the galvanic and auditory stimulations.

The optional detectors 14 for directly or indirectly detecting respiratory arrest may also be incorporated into the headband, retained at one or more pockets on the headband, or located off the headband and coupled to the microprocessor 16, via either wires or wireless communication. The microprocessor 16 and power supply 18 alternatively may be located external of the headband 20 and connected to the stimulators by wires.

The system is preferably worn by the user and operated, in accord with the method described below, to prevent or interrupt persistent apnea using stimuli applied via the system. The system is adapted to have a sufficient vestibular stimulatory effect on the user to prevent terminal respiratory arrest, while also allowing the user to remain sleeping. The stimulation of the vestibular system can prevent or treat persistent respiratory arrest including reversing or preventing vocal cord closure.

In conjunction with the headband, the system can include an oxygen delivery system.

In accord with one method of using the system, the system is applied to the user. As indicated above, the system is adapted to be worn by the user such that a pair of galvanic stimulating electrodes 112a are applied over the bones at the left and right mastoid processes of the user.

The method and system are directed to stimulate the vestibular system to reverse the brainstem activity otherwise initiating diving response. The effect of such a reversal is restoration of breathing effort and, provided the airway is clear, effective respiration being restored.

The method and system include one or more options to activate the vestibular system, including galvanic vestibular stimulation and low frequency sound wave stimulation preferably via bone conduction which stimulates the vestibular system. Bone conduction is particularly advantageous for sound stimulation as individuals prone to sudden death syndromes are susceptible to having fluid or pus in the middle ear impeding air conducted sound to the inner ear. Such methods provide effective activation of the vestibular system to prevent respiratory arrest when used prophylactically, as well as to restart breathing after the point of respiratory arrest when used as a treatment upon indication of breathing irregularity leading up to arrest. The methods may also include delivery of oxygen to prevent breathing arrest and/or restart arrested breathing.

In an embodiment, the method and use of the system is implemented without reliance upon sensors that detect respiratory arrest, and a level of vestibular stimulation is provided at regular intervals to prevent or overcome respiratory arrest.

Experimental results from animal studies validate the concepts of using vestibular stimulation in the manner discussed and applied herein. Tests were performed on adult rat subjects. Turning to FIG. 4, in a first pre-test experiment on a rat subject, trace 302 shows pressure transients, and trace 312 is an electrocardiogram (EKG) reading of the same subject over the same time period, in seconds along the x-axis. The y-axis of for trace 312 represents tracheal pressure in mmHg*10; the y-axis for trace 312 is in mV. After a baseline period of normal tidal breathing during 304, 314, the subject's airway was artificially occluded to cause a period of obstructive apnea. The time during which the airway was occluded is indicated at 306 by a series of progressively larger negative transients, representing attempts to draw breaths against the closed airway. The breath attempts stop at 308, signifying the moment of respiratory arrest. No further spontaneous breathing attempts are made. This point of respiratory arrest is comparable to the onset of the a response. The subject is manually resuscitated via a series of lung inflations at 310, and breathing then restarts at 311, 321.

In a second test on the rat subject, a 50 Hz sine wave (2V peak to peak output from a sine wave generator) was provided directly to the subject's mastoid process using a B81 bone conductor (Merz Medizentechnik GmbH) and operated to apply the 50 Hz to the subject via bone conduction throughout the experiment. The experiment is shown at traces 332, 342, in which trace 332 identifies pressure transients, and trace 342 identifies EKG readings. After a baseline period of normal tidal breathing during 334, 344, the subject's airway was artificially occluded to cause a period of obstructive apnea. The time during which the airway was occluded is indicated at 336 by a series of large negative transients, representing attempts to draw breaths against the closed airway. The breath attempts stop at 338, signifying respiratory arrest. While breathing did not spontaneously resume, only two recovery breaths, at 340, were required to manually resuscitate; the recovery breaths are also generally larger in amplitude than in the control experiment.

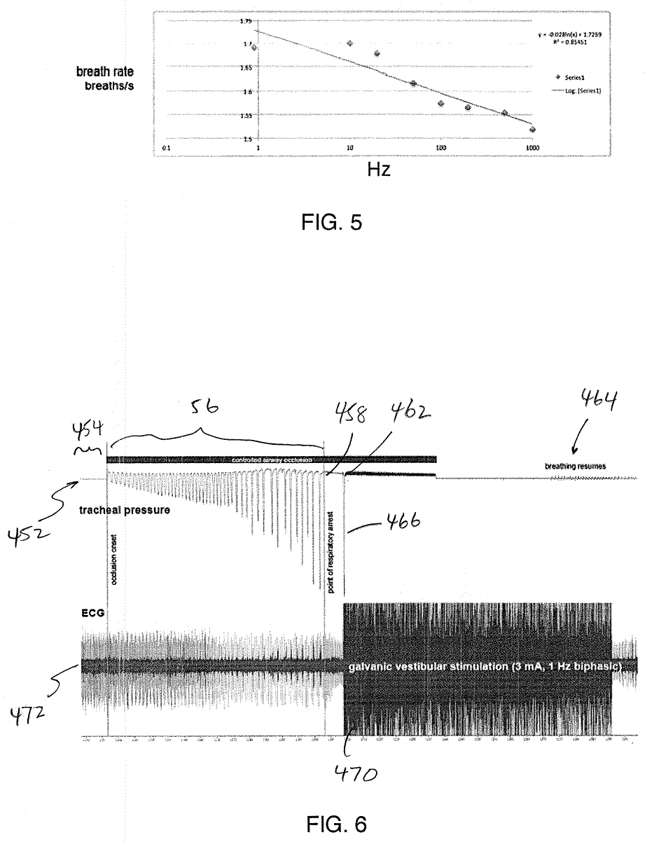

As indicated, the data for the prior experiment provides results of applied 50 Hz continuous sine wave only. Other frequencies have been tested for their effect on breathing rate. Turning to FIG. 5, the breath rate (breaths/seconds) is shown for experimental data of applied sine wave frequencies from 10 Hz to 1000 Hz. The breathing rate is shown to decrease as the stimulus frequency is increased. The data plot shows that 50 Hz is approximately a threshold for a measurable change in breathing frequency and, from FIG. 4, that 50 Hz stimulation facilitated resuscitation. However, in the experiments on the rat subjects, 50 Hz stimulation did not resuscitate by itself when initiated after the point of respiratory arrest or when applied continuously.

In a second test, galvanic vestibular stimulation was scaled for application to a rat subject. A rubber headband retained two copper electrode contacts at a stable position against the mandibular joint. A conductive gel was applied to the electrodes and permitted electrical connection through the fur and skin such that applied conduction was made to the underlying bone. Continuous pulses of 3 mA current at alternating 0.5 second phases (square pulses) was applied to the electrodes after the animal had passed to point of respiratory arrest. The results of the test are shown at FIG. 6 in which trace 452 indicates tracheal pressure and trace 472 indicates an EKG reading. Similar to the prior test, after a baseline period of normal tidal breathing during 454, the subject's airway was artificially occluded at 456 to cause a period of obstructive apnea. The occlusion 456 is indicated by a series of large negative transients, representing attempts to draw breaths against the closed airway. The breath attempts stop at 458, signifying respiratory arrest. Galvanic stimulation was started at 462, 470. Breathing resumes spontaneously at 464 after respiratory arrest as a result of the galvanic vestibular stimulation. It is also noted that one breath attempt was made earlier at 466, as soon as galvanic stimulation was applied but before the airway was cleared; it is believed that breathing would have restarted at this earlier time if the airway would have been clear.

There have been described and illustrated herein embodiments of systems and methods for the prevention and treatment of sudden death syndromes. While particular embodiments of the invention have been described, it is not intended that the invention be limited thereto, as it is intended that the invention be as broad in scope as the art will allow and that the specification be read likewise. Thus, while particular stimulators, detectors, and sensors have been disclosed, it will be appreciated that other suitable stimulators and detectors can be used as well. In addition, while particular devices for coupling the stimulators to the body of the user for transmitting the stimulation to the vestibular system have been disclosed, it will be understood other suitable devices can be used. It will therefore be appreciated by those skilled in the art that yet other modifications could be made to the provided invention without deviating from its scope as claimed.

What is claimed is:

1. A system for the prevention of apnea during a sleeping state of a user, comprising:
a) a galvanic vestibular stimulator;
b) an audio vestibular stimulator;
c) a wearable garment adapted to retain at least portions of the galvanic vestibular stimulator and the audio vestibular stimulator relative to the user; and
d) a power supply to power the galvanic vestibular stimulator and the audio vestibular stimulator.

2. The system of claim 1, wherein the galvanic vestibular stimulator includes a pair of electrodes and a current generator that is adapted to generate a wave of alternating stimulus phases between the pair of the electrodes.

3. The system of claim 2, wherein the current generator is adapted to generate a square wave, triangle wave, or sine wave.

4. The system of claim 2, the pair of electrodes is adapted for retention against an external surface of the user.

5. The system of claim 4, wherein the electrodes include at least one of conductive gel pads and adhesive pads.

6. The system of claim 1, wherein the audio vestibular stimulator includes a low frequency audio generator that is adapted to generate audio or vibrational frequencies below 250 Hz and a pair of non-implantable transducers adapted to transmit audio or vibrational frequencies below 250 Hz.

7. The system of claim 6, wherein the transducers are adapted to transmit audio or vibrational frequencies below 250 Hz audio to the user via bone conduction.

8. The system of claim 6, wherein the transducers are adapted to further transmit frequencies between 250 Hz and 20,000 Hz.

9. The system of claim 1, further comprising:
a detector to detect when the user is subject to breathing arrest,
the detector coupled relative to at least one of the galvanic vestibular stimulator and the audio vestibular stimulator such that, when breathing arrest is detected, at least one of the galvanic vestibular stimulator and the audio vestibular stimulator is activated to provide a stimulation to the user.

10. The system of claim 9, wherein the detector includes at least one of electroencephalogram sensors, otoacoustic emission sensors, and vestibular evoked myogenic potential sensors.

11. The system of claim 9, wherein the detector includes at least one of oxygen status sensors such as pulse oximeters or near infrared spectroscopy sensors to detect blood oxygen levels, transcutaneous carbon dioxide sensors, and temperature sensors.

12. A system for the prevention of apnea in a user during a sleeping state, the user having a head with a vestibular system, comprising:
a vestibular stimulator including a pair of non-implantable stimulators for retention on the head, the vestibular stimulator actuatable to sufficiently stimulate the vestibular system of the user to prevent the user from having breathing arrest.

13. The system of claim 12, further comprising a gyroscope that detect changes in a position of the head, wherein feedback from the gyroscope results in adjustment to stimulation from at least one of the pair of non-implantable stimulators.

14. The system of claim 12, wherein the stimulators are adapted to stimulate the vestibular system of the user to prevent the user from having breathing arrest without waking the user from the sleeping state.

15. A system for a user vulnerable to breathing arrest, the user having a head with a vestibular system, comprising:
a) a vestibular system stimulator including a pair of non-implantable stimulators for retention on the head, the vestibular system stimulator actuatable to provide a first level of stimulation to the vestibular system; and
b) a detector to detect when the user is subject to breathing arrest, the detector coupled relative to the vestibular stimulator such that if breathing arrest is detected, the vestibular stimulator is activated to provide a higher second level of stimulation to the vestibular system.

16. The system of claim 15, wherein the first level of stimulation is adapted to be below a threshold which would wake the user from a sleeping state.

17. A method of preventing permanent breathing arrest in a user, comprising:
stimulating a vestibular system of the user via at least one of,
electrical galvanic vestibular stimulation, and
sound and/or vibration-induced vestibular stimulation using bone conduction.

18. The method of claim 17, wherein the stimulating occurs via both of electrical galvanic vestibular stimulation and sound-induced vestibular stimulation.

19. The method of claim 17, wherein the stimulating includes electrical galvanic vestibular stimulation applied by a current passed between a pair of electrodes non-invasively positioned at or adjacent left and right mastoid bones on a head of the user.

20. The method of claim 17, wherein the stimulating includes sound and/or vibration-induced vestibular stimulation applied by at least one non-implantable transducer positioned on the head or in an ear and passing sound or vibration to the vestibular system.

21. The method of claim 20, wherein the transducer generates a frequency below 250 Hz.

22. The method of claim 20, wherein the transducer generates a frequency that is infrasonic.

23. The method of claim 17, further comprising:
identifying if breathing arrest occurs in the user, and
if breathing arrest occurs, then stimulating the vestibular system of the user with a higher level of stimulation via at least one of,
electrical galvanic vestibular stimulation, and
sound and/or vibration-induced vestibular stimulation.

24. The method of claim 17, wherein the vestibular system is stimulated in an individual susceptible to a sudden death syndrome.
